(19) United States
(12) Patent Application Publication
(10) Pub. No.: US 2016/0113581 A1
(43) Pub. Date: Apr. 28, 2016

(54) DOCKING STATION FOR SMART GARMENTS

(71) Applicant: HEALTHWATCH LTD., Herzliya (IL)

(72) Inventors: Uri AMIR, Or Yehuda (IL); Boaz Shoshani, Raanana (IL)

(73) Assignee: HEALTHWATCH LTD., Herzliya (IL)

(21) Appl. No.: 14/895,801

(22) PCT Filed: Jun. 7, 2014
(86) PCT No.: PCT/IL2014/050512

§ 371 Date: Dec. 3, 2015

Related U.S. Application Data
(60) Provisional application No. 61/832,163, filed on Jun. 7, 2013.

(51) Int. Cl. A61B 5/00; A41D 13/12; A61B 5/04; A41D 1/00

(52) U.S. Cl. CPC A61B 5/6804; A41D 1/005; A41D 13/1281; A61B 5/04

(57) ABSTRACT

A monitoring system for monitoring physiological parameters of a living being. The monitoring system includes a smart garment, a processing unit and a docking station. The docking station enables quick and easy engagement/disengagement of the processor. The docking station is electrically connected to sensors of the smart garment to thereby provide the sensed data to the processor that is docked thereon. The processor is adapted to obtain some or all of the sensed data via a communication interface such as HDMI. The docking station and the smart garment may be interconnected using complementary conductive snap buttons. The docking station may include at least one through opening formed in its back wall, to thereby enable wired operational communication between the processing unit and a respective additional external sensor. The docking station may include a defibrillator protection device to protect the monitoring system from an electric current or voltage surge.

### CROSS REFERENCE TO RELATED APPLICATIONS

This application claims the benefit under 35 USC 119(e) from U.S. provisional application 61/832,163 filed Jun. 7, 2013, the disclosures of which is included herein by reference.

This application also related to PCT application PCT/IL2013/050963, filed Nov. 23, 2013, entitled “Vertical conductive textile traces and methods of knitting thereof”, PCT application PCT/IL2013/050964, filed Nov. 23, 2013, entitled “Float loop textile electrodes and methods of knitting thereof”, and non-published U.S. provisional application 61/950,139 filed Mar. 9, 2014, all of which are incorporated herein by reference as if fully set forth herein

### FIELD OF THE INVENTION

The present invention relates to health monitoring systems and more particularly, the present invention relates to a docking station for a smart garment, being a monitoring system of a living being wearing the smart garment. The docking station facilitates hosting various types of processing units for monitoring a variety of physiological parameters of the living being.

### BACKGROUND OF THE INVENTION AND PRIOR ART

Smart garments are designed to monitor living beings wearing the smart garment. A smart garment may include a variety of textile sensors and other types of sensors for detecting different physiological and chemical parameters of the living being. Smart garments also include a processor for analyzing the sensed data. Typically, the textile sensors have conductive textile traces that facilitate transmitting the sensed data from the textile sensors to the processor.

An example smart garment is described in PCT application PCT/IL2012/000248, filed on Sep. 20, 2010, entitled “Continuous non-interfering health monitoring and alert system, which is incorporated herein by reference as if fully set forth herein.

Typically, the smart garment is coupled to operate with various types of processors, for analyzing different physiological parameters of the living being. Some processors may be used at normal daytime activity, at night, while performing sport activities, while swimming, etc. Also, there is often a need for quick engagement/disengagement of the processor, for example, when washing the garment. To facilitate quick engagement/disengagement of the processor, a docking station may be used. Preferably, the engagement/disengagement is easy to perform, as the user may be a disabled person, old and sick, and sometimes with no help of a caretaker.

There is a need that the processor is be stable while being worn with the garment, and that the docking station is light weight, narrow (so the complex docking station-processor does not standing out) and water durable as it might be machine washed with the garment.

### BRIEF SUMMARY OF THE INVENTION

The principal intentions of the present invention include providing a docking station for a smart garment, facilitating quick and easy engagement/disengagement of the processor. The docking station is electrically connected to the conductive traces and provides the sensed data to the processor that is docked thereon. The processor is adapted to obtain some or all of the sensed data.

According to the teachings of the present invention, there is provided a monitoring system for monitoring physiological parameters of a living being. The monitoring system includes a smart garment, a processing unit and a docking station.

The smart garment includes sensors for sensing electric vital signals of the living being, first-wiring connecting devices and garment-electric-wiring-means, electrically interconnecting to the sensors with respective first-wiring-connecting devices.

The docking station includes a body adapted to receive the processing unit, the body having a back wall with an external surface and an internal surface. The docking station further includes a socket structure, docking-station-wiring-means and second-wiring-connecting devices enabling the transfer of the sensed vital signals. The processing unit is adapted to receive and process the sensed vital signals.

Each of the first-wiring-connecting devices is adapted to interconnect with the respective second-wiring-connecting device, thereby allowing transfer of the sensed vital signals from the sensors to the docking station. The docking-station-wiring-means are individually preconfigured to further transfer the sensed vital signals from the docking station to the processing unit.

The socket structure is attached to the internal surface of the back wall, protruding away from the monitored living being, wherein the socket structure includes a first side wall, a second side wall, and a seat. The seat interconnects a first end of the first side wall and a first end of the second side wall, to form a generally U-shaped structure being an operational seating bench for the processing unit.

The processing unit includes a first side wall, a second side wall and a landing side, wherein the first side wall of the processing unit includes a first receiving groove and the second side wall of the processing unit includes a second receiving groove. On the internal side of the first side wall, the first side wall includes a first rail rib, protruding inwardly and forming a first groove between the first rail rib and internal surface of the back wall. On the internal side of the second side wall, the second side wall includes a second rail rib protruding inwardly and forming a second groove between the second rail rib and internal surface of the back wall. The first rail rib is adapted to fittingly receive the first receiving groove, the first rail rib is adapted to fittingly receive the first receiving groove and the seat is adapted to operatively receive the landing side of the processing unit.

The seat and the landing side include a communication interface facilitating operational communication flow between the docking station and the processing unit and thereby also facilitating operational communication flow between the sensors and the processing unit. The communication interface is selected from the group including, with no limitations, USB and HDMI.

Preferably, the docking station further includes a locking and release mechanism adapted to lock the processing unit to the docking station lock, when in operation, and to disengage the processing unit from the docking station, when desired. Upon insertion of the processing unit, the locking and release mechanism locks the processing unit in operational sitting position inside the socket structure.

Preferably, with no limitations, the sensors are textile electrodes selected from the group including knitted electrodes and interwoven electrodes.

Optionally, the garment-electric-wiring-means and the docking-station-wiring-means are detachably interconnected.

Optionally, the first-wiring connecting devices and the second-wiring connecting devices are complementary conductive snap buttons.

Preferably, each of the first-wiring connecting devices is attached to a respective garment-electric-wiring-means.

Optionally, each second-wiring connecting device is disposed at a preconfigured location of the external surface of the back wall and is operatively attached to a respective docking-station-wiring-means.

Optionally, each line of the docking-station-wiring-means includes a defibrillator-protection-device to protect the monitoring system from an electric current or voltage surge, such as during using a defibrillator.

Optionally, the docking station further includes at least one through opening formed in the back wall, thereby enabling wired operational communication between the processing unit and a respective additional sensor, being external to the smart garment.

Preferably, the docking station is made of materials enabling machine washing, such as plastic.

According to further teachings of the present invention, there is provided a docking station adapted to receive a processing unit adapted to receive and process at least one signal from a respective sensor. The docking station includes a body adapted to receive the processing unit, wherein the body includes a back wall with an external surface and an internal surface.

The docking station further includes a socket structure, docking-station-wiring-means and a connecting device for each of the sensors, enabling transfer of the respective sensed signal. Each of the sensors is adapted to operatively connect with the respective connecting device, thereby allowing transfer of the sensed signal from said at least one sensor to the docking station. The docking-station-wiring-means are individually preconfigured to further transfer the respective signals from the docking station to the processing unit.

### BRIEF DESCRIPTION OF THE DRAWINGS

The present invention will become fully understood from the detailed description given herein below and the accompanying drawings, which are given by way of illustration and example only and thus not limitative of the present invention:

FIG. 1 illustrates a docking station for a smart garment, hosting a processing unit coupled to operate with sensing devices embedded in the smart garment, according to embodiments of the present invention.

FIG. 2 is an exploded view illustration of the docking station and processing unit shown in FIG. 1.

FIG. 3a is a first side perspective view illustration of the docking station shown in FIG. 1.

FIG. 3b is a second side perspective view illustration of the docking station shown in FIG. 1.

FIG. 4a is a first side perspective view illustration of the processing unit shown in FIG. 1.

FIG. 4b is a second side perspective view illustration of the processing unit shown in FIG. 1.

FIG. 5 is a rear perspective view illustration of the docking station shown in FIG. 1.

FIG. 6 is a top view illustration of the docking station shown in FIG. 1.

FIG. 7 schematically illustrates an example smart garment, having a docking station interface, according to embodiments of the present invention.

FIG. 8 schematically illustrates an example smart garment, as shown in FIG. 6, having a docking station, as shown in FIG. 1, attached thereto.

FIG. 9 schematically illustrates an example electric diagram, having defibrillator protection devices, according to embodiments of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and material and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

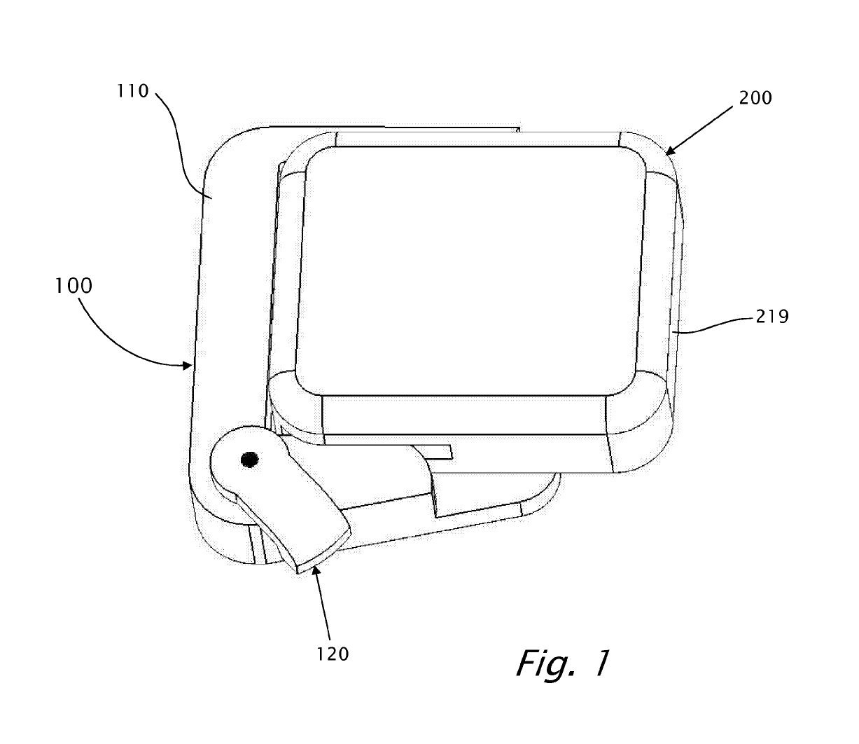

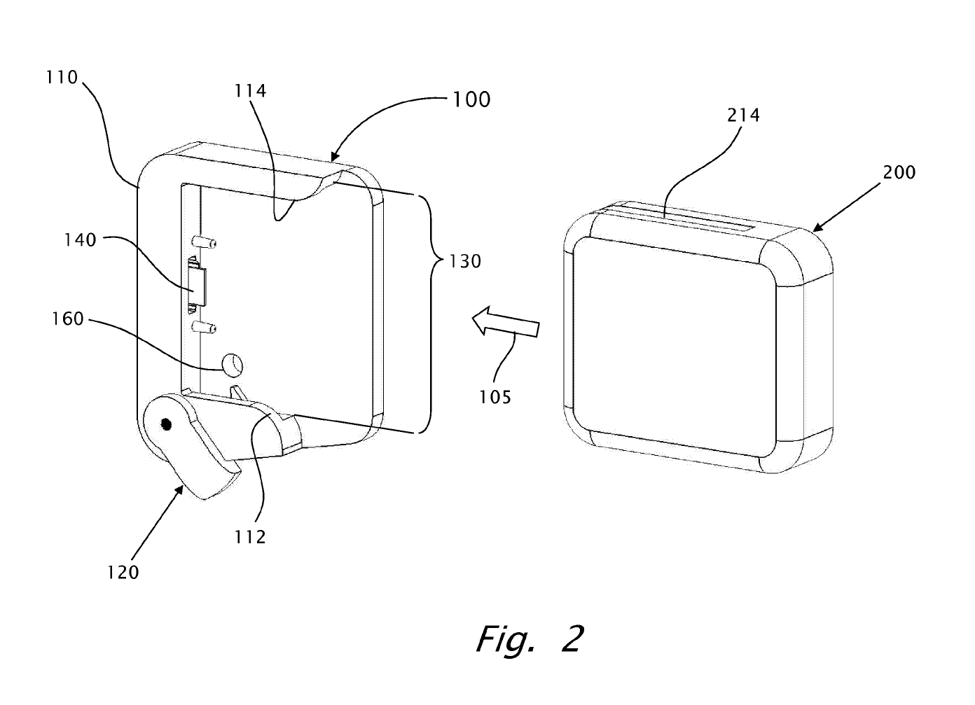

Reference now made to the drawings. FIG. 1 illustrates a docking station 100 for a smart garment, hosting a processing unit 200 coupled to operate with sensing devices embedded in the smart garment, according to embodiments of the present invention, processing unit 200 being an exemplary processing unit. FIG. 2 is an exploded view illustration of docking station 100 and processing unit 200, showing the mounting direction 105.

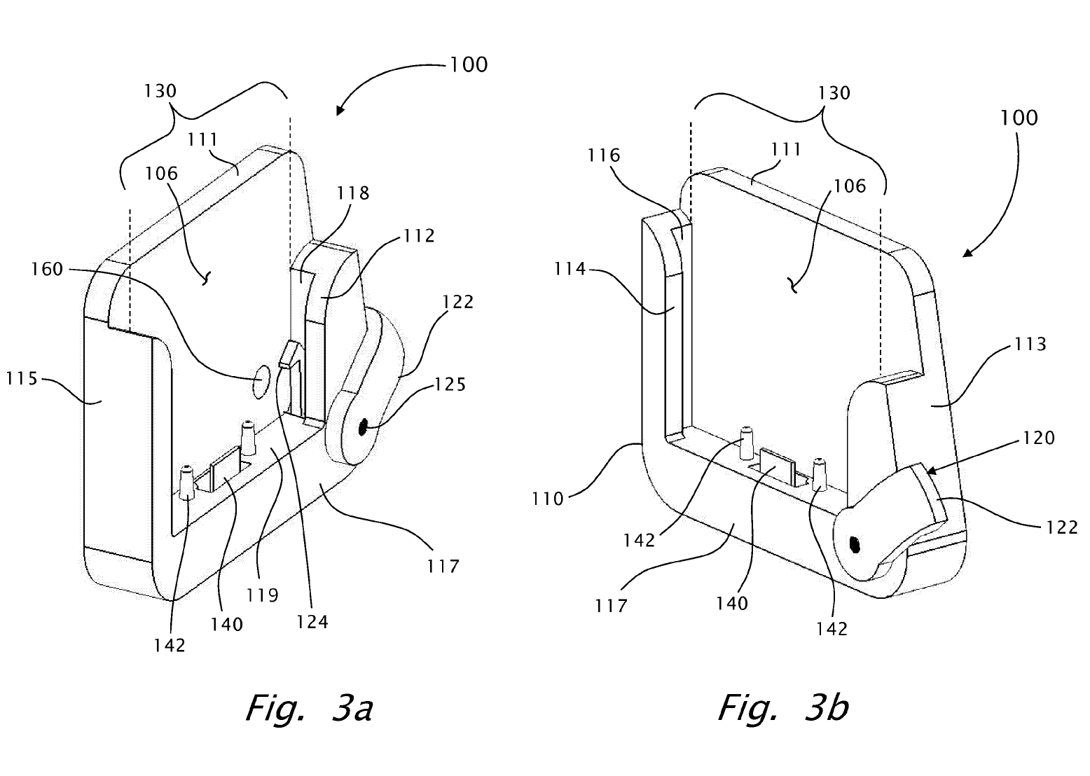

FIG. 3a is a first side perspective view of docking station 100 and FIG. 3b is a second side perspective view of docking station 100. Docking station 100 includes a body 110 having a back wall 111 with an external surface and an internal surface 106, a socket structure, docking-station-wiring-means and preferably, a locking and release mechanism 120.

The socket structure is composed of a first side wall 113, a second side wall 115 and a seat 117. Typically, seat 117 interconnects a first end of first side wall 113 and a first end of second side wall 115, to form to form a generally U-shaped structure being an operational seating bench for processing unit 200. On the internal side, first side wall 113 includes a rail rib 112, protruding inwardly from the internal side of first side wall 113. A groove 118 is formed between rail rib 112 and internal surface 106 of back wall 111. On the internal side, second side wall 115 includes a rail rib 114, protruding inwardly from the internal side of second side wall 115. A groove 116 is formed between rail rib 114 and internal surface 106 of back wall 111. In operational mode, back wall 111 serves as a partition between said processing unit 200 and the garment/user. First side wall 113 is opposing second side wall 115, wherein the lateral distance 130 between groove 118 and groove 116 facilitates a fitted insertion of processing unit 200 into the socket structure of docking station 100.

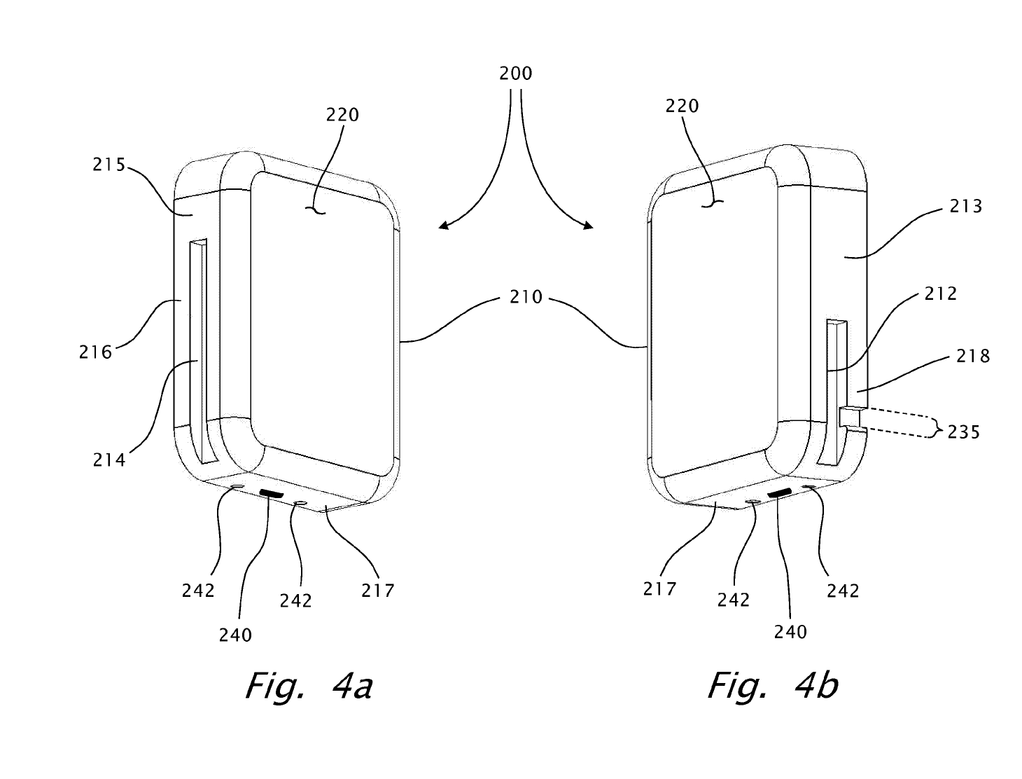

FIG. 4a is a first side perspective view of processing unit 200 and FIG. 4b is a second side perspective view of processing unit 200. Processing unit 200 includes a housing 210 having a front face 220, a rear face (not shown, typically parallel to front face 220), a first side wall 213, a second side wall 215, a top side 219 and a landing side 217. The front face 220 is opposing the rear face, first side wall 213 is opposing second side wall 215 and top side 219 is opposing landing side 217. When processing unit 200 is inserted into the socket structure of docking station 100, landing side 217 is disposed adjacently to the internal side 119 of seat 117.

A groove 212 is formed in first side wall 213 extending all the way towards landing side 217. At least one rib 218 is formed by groove 212 between groove 212 and the rear face of processing unit 200. A groove 214 is formed in second side wall 215 extending all the way towards landing side 217. At least one rib 216 is formed by groove 214 between groove 214 and the rear face of processing unit 200.

The lateral distance 130 between groove 118 and groove 116 facilitates the insertion of processing unit 200 into the socket structure of docking station 100, wherein the lateral distance between rib 218 and rib 216 is fitted to lateral distance 130. To insert processing unit 200 into the socket structure of docking station 100 (in direction 105), rib 216 is slidingly inserted into groove 116 and rib 218 is slidingly inserted into groove 118.

In one embodiment, locking and release mechanism 120 includes a release handle 122, operatively connected to a latch 124, by a shaft 125. When a user inserts processing unit 200 into the socket structure of docking station 100 (in direction 105), latch 124 is pushed back rotating shaft 125 backwards against a biasing element such as a spring (not shown). When landing side 217 reaches seat 117, latch 124 is pushed forward by the biasing element and into a designated lateral groove 235, formed in rib 218 in a preconfigured location.

To release processing unit 200 from the socket structure of docking station 100, the user pivots release handle 122, overcoming the force embedded in the biasing element, to thereby move latch 124 out of lateral groove 235, thereby enable processing unit 200 to slide out of the socket structure of docking station 100.

It should be noted that locking and release mechanism 120 is given by way of example only and other lock-and-release mechanisms known in the art may be use.

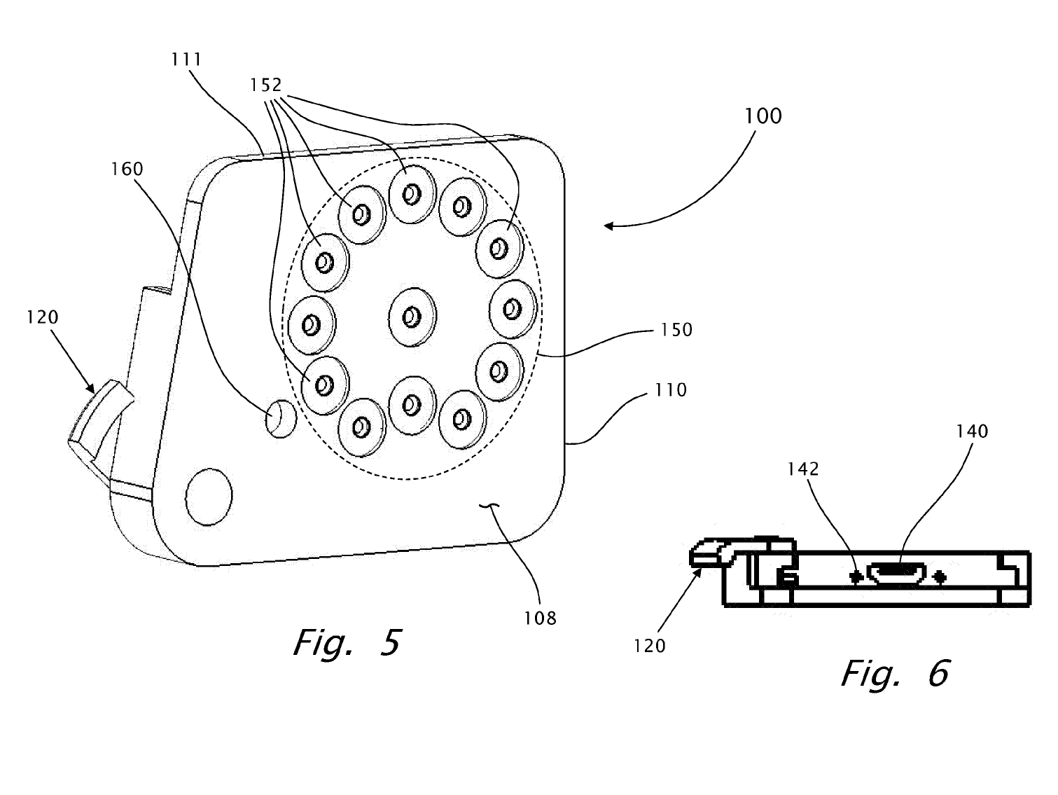

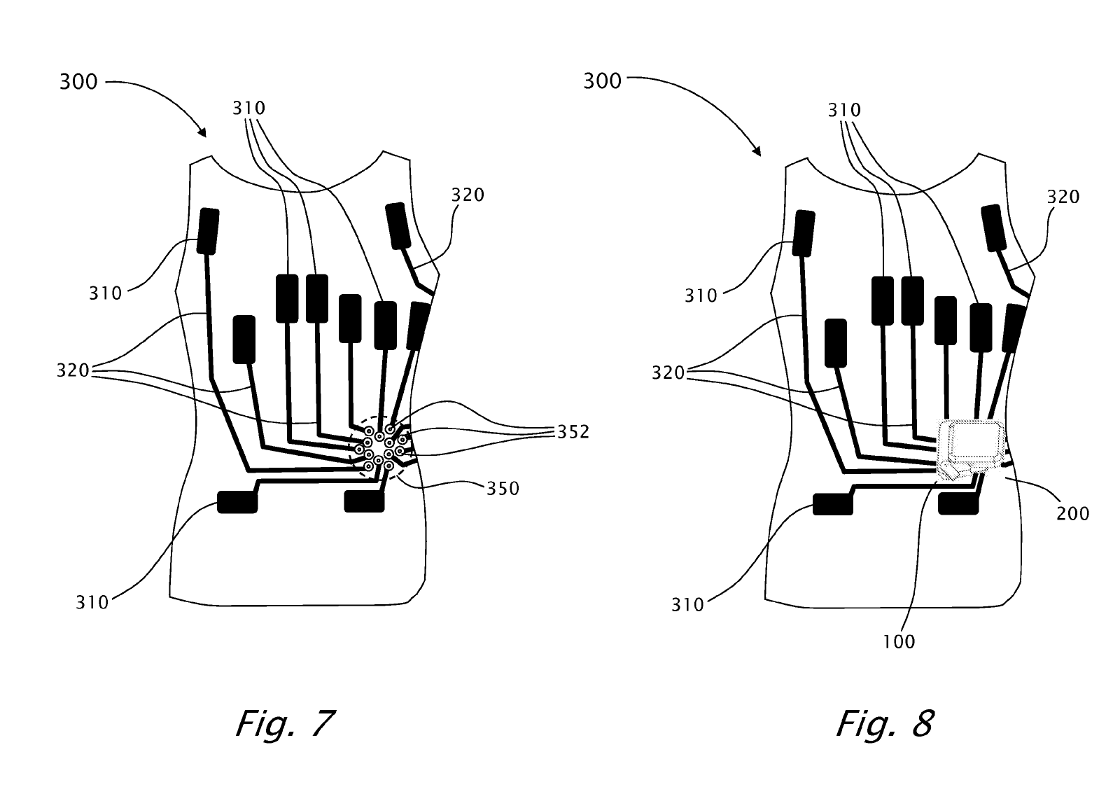

Reference is now made to FIG. 5, a rear perspective view of docking station 100; and to FIG. 6, a top view of docking station 100. Reference is also made to FIG. 7 that schematically illustrates an example smart garment 300, having a docking station interface 350, according to embodiments of the present invention; and to FIG. 8, a schematic illustration of smart garment 300, as shown in FIG. 7, having a docking station 100, as shown in FIG. 1, attached thereto.

The external surface 108 of back wall 111 of docking station 100 includes a garment interface 150, according to embodiments of the present invention. Garment interface 150 is designed to operatively interface processing unit 200 with at least one electrical signal measured by a sensor knitted into smart garment 300. Smart garment 300 includes multiple knitted sensors 310, conductive traces 320 and docking station interface 350.

In one embodiment of the present invention, garment interface 150 and docking station interface 350 include conductive snap buttons 152 and 352, respectively. Each snap buttons 352 is connected to a specific conductive trace 320 that transfers an electric signal from a respective sensor 310, typically knitted textile sensor 310, to processing unit 200 that is docked into the socket structure of docking station 100, through snap buttons 352 and the respective snap buttons 152 snapped there onto.

Docking station 100 is preconfigured with a signal outlet for each snap buttons 152, and processing unit 200 has respective signal inlets on body 210 that are aligned with the corresponding signal outlet of docking station 100. In the example shown in the Figures, an HDMI interface is illustrated, with no limitation. Any other communication interface known in the art, such as USB, may be used. In the example Figures, docking station 100 includes an HDMI outlet 140 and processing unit 200 has respective signal inlet 240. Respective anchoring leading devices 142 and 242 may be used to ease the HDMI connecting process.

Hence, an electric signal sensed by a sensor 310 is transferred via a respective conductive trace 320 to a preconfigured snap buttons 352. The signal is then transferred via the respective snap buttons 152 that is snapped onto that snap buttons 352, via docking-station-wiring-means (not shown), typically wired inside docking station 100, to a preconfigured pin of the communication plug interface, wherein in the example shown, the communication plug interface is HDMI outlet 140. From HDMI outlet 140 the sensed signal reached its destination, being processing unit 200, via HDMI inlet 240 that is operatively engaged with HDMI outlet 140.

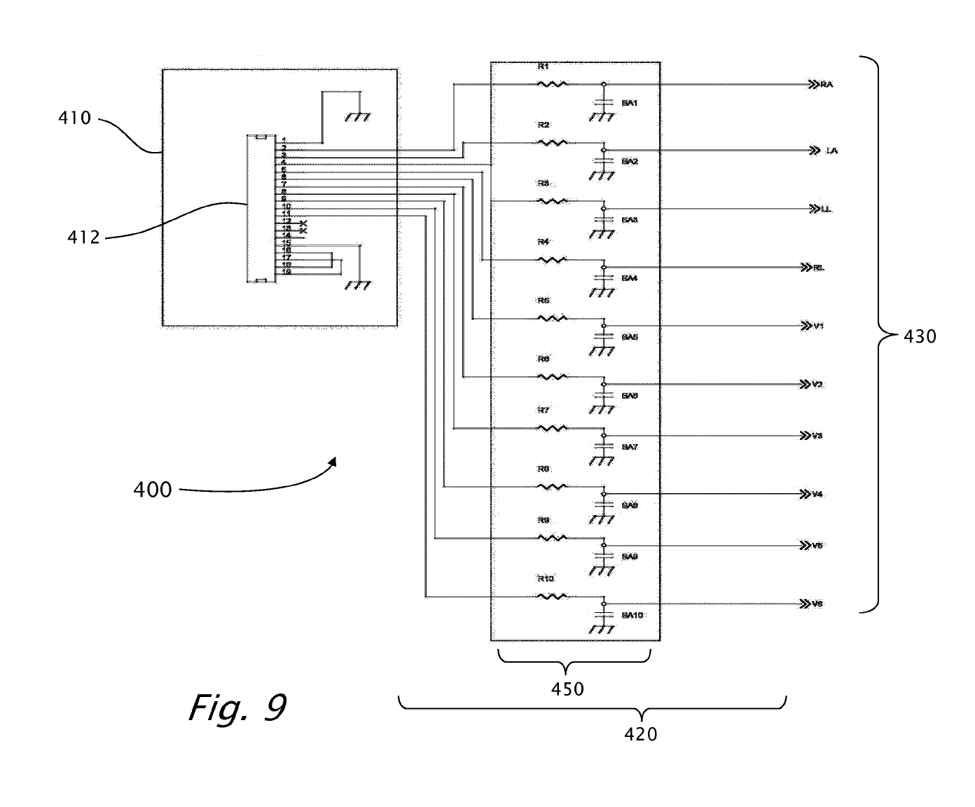

Optionally, each line of the docking-station-wiring-means includes a defibrillator-protection-device to protect the monitoring system from an electric current or voltage surge, such as during using a defibrillator. FIG. 9 schematically illustrates an example electric diagram 400 of a docking station 100, having defibrillator protection devices 450, according to embodiments of the present invention. In the example shown in FIG. 9, electric diagram 400 includes a HDMI connector box 410 having a HDMI connector 412, docking-station-wiring-means 420 including respective protection devices 450 for each lead 430. Defibrillator protection devices are known in the art and any type of defibrillator protection devices may be used, for example: Zener diodes.

Docking station 100 may further include at least one through opening 160 formed in back wall 111, thereby enabling wired operational communication between processing unit 200 and a respective additional sensor, being external to smart garment 300.

It should be noted that conductive snap buttons 152 and 352 are given by way of example only and with no limitations, and any other detachable conductive wiring connecting devices may be used.

It should be further noted that conductive traces 320 are given by way of example only and other electric wiring known in the art, such as conductive stripes (see U.S. provisional application 61/950,139), may be used.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A monitoring system for monitoring physiological parameters of a living being, comprising:
a) a smart garment comprising:
i. sensors for sensing electric vital signals of the living being;
ii. first-wiring connecting devices; and
iii. garment-electric-wiring-means electrically interconnecting to said sensors with respective said first-wiring-connecting devices;
b) a processing unit adapted to receive and process the sensed vital signals; and
c) a docking station comprising:
i. a body adapted to receive said processing unit, said body having a back wall with an external surface and an internal surface;
ii. a socket structure;
iii. docking-station-wiring-means; and
iv. second-wiring-connecting devices enabling transfer of the sensed vital signals,
wherein each said first-wiring-connecting device is adapted to interconnect with respective said second-wiring-connecting device, thereby allowing transfer of the sensed vital signals from said sensors to said docking station;
wherein said docking-station-wiring-means are individually preconfigured to further transfer the sensed vital signals from said docking station to said processing unit.

2. The monitoring system of claim 1, wherein said socket structure is attached to said internal surface of said back wall, protruding away from the monitored living being.

3. The monitoring system of claim 2, wherein said socket structure comprises:
a) a first side wall;
b) a second side wall; and
c) a seat,
wherein said seat interconnects a first end of said first side wall and a first end of said second side wall, to form a generally U-shaped structure being an operational seating bench for said processing unit.

4. The monitoring system of claim 3, wherein said processing unit includes a first side wall, a second side wall and a landing side,
wherein said first side wall of said processing unit comprises a first receiving groove and said second side wall of said processing unit comprises a second receiving groove;
wherein on the internal side of said first side wall, said first side wall comprises a first rail rib protruding inwardly and forming a first groove between said first rail rib and internal surface of said back wall;
wherein on the internal side of said second side wall, said second side wall comprises a second rail rib protruding inwardly and forming a second groove between said second rail rib and internal surface of said back wall; and
wherein said first rail rib is adapted to fittingly receive said first receiving groove, said first rail rib is adapted to fittingly receive said first receiving groove and said seat is adapted to operatively receive said landing side of said processing unit.

5. The monitoring system of claim 1, wherein said seat and said landing side comprise a communication interface facilitating operational communication flow between said docking station and said processing unit and thereby also facilitating operational communication flow between said sensors and said processing unit.

6. The monitoring system of claim 5, wherein said communication interface is selected from the group including USB and HDMI.

7. The monitoring system of claim 1, wherein said docking station further comprises a locking and release mechanism adapted to lock said processing unit to said docking station lock, when in operation, and to disengage said processing unit from said docking station, when desired.

8. The monitoring system of claim 7, wherein upon insertion of said processing unit, said locking and release mechanism locks said processing unit in operational sitting position inside said socket structure.

9. The monitoring system of claim 1, wherein said sensors are textile electrodes selected from the group including knitted electrodes and interwoven electrodes.

10. The monitoring system of claim 1, wherein said garment-electric-wiring-means and said docking-station-wiring-means are detachably interconnected.

11. The monitoring system of claim 1, wherein said first-wiring connecting devices and said second-wiring connecting devices are complementary conductive snap buttons.

12. The monitoring system of claim 1, wherein each said first-wiring connecting device is attached to a respective garment-electric-wiring-means.

13. The monitoring system of claim 1, wherein each said second-wiring connecting device is disposed at a preconfigured location of said external surface of said back wall and is operatively attached to a respective docking-station-wiring-means.

14. The monitoring system of claim 1, wherein each line of said docking-station-wiring-means includes a defibrillator-protection-device to protect said monitoring system from an electric current or voltage surge, such as during using a defibrillator.

15. The monitoring system of claim 1, wherein said docking station further comprises at least one through opening formed in said back wall, thereby enabling wired operational communication between said processing unit and a respective additional sensor, being external to said smart garment.

16. The monitoring system of claim 1, wherein said docking station is made of materials enabling machine washing, such as plastic.

17. A docking station adapted to receive a processing unit adapted to receive and process at least one signal from a respective sensor, the docking station comprising:
a) a body adapted to receive said processing unit, said body having a back wall with an external surface and an internal surface;
b) a socket structure;
c) docking-station-wiring-means; and
d) a connecting device for each said sensor enabling transfer of the respective sensed signal,
wherein each said sensor is adapted to operatively connect with respective said connecting device, thereby allowing transfer of the sensed signal from said at least one sensor to said docking station;
wherein said docking-station-wiring-means are individually preconfigured to further transfer the respective signals from said docking station to said processing unit.
